SEQUENCE LISTING (51 sequences)

SEQ ID NO: 1 - protein, 8 aa, Artificial Sequence
  Synthetic
XXXXXXXX

SEQ ID NO: 2 - protein, 7 aa, Artificial Sequence
  Synthetic
RVYIHPF

SEQ ID NO: 3 - protein, 6 aa, Artificial Sequence
  Synthetic
VYIHPF

SEQ ID NO: 4 - protein, 7 aa, Artificial Sequence
  Synthetic
DRVYIHP

SEQ ID NO: 5 - protein, 6 aa, Artificial Sequence
  Synthetic
RVYIHP

SEQ ID NO: 6 - protein, 5 aa, Artificial Sequence
  Synthetic
VYIHP

SEQ ID NO: 7 - protein, 4 aa, Artificial Sequence
  Synthetic
IHPF

SEQ ID NO: 8 - protein, 6 aa, Artificial Sequence
  Synthetic
DRVYIH

SEQ ID NO: 9 - protein, 5 aa, Artificial Sequence
  Synthetic
DRVYI

SEQ ID NO: 10 - protein, 4 aa, Artificial Sequence
  Synthetic
DRVY

SEQ ID NO: 11 - protein, 7 aa, Artificial Sequence
  Synthetic
RLYIHPF

SEQ ID NO: 12 - protein, 7 aa, Artificial Sequence
  Synthetic
RVYLHPF

SEQ ID NO: 13 - protein, 8 aa, Artificial Sequence
  Synthetic
DRPYIHPF

SEQ ID NO: 14 - protein, 7 aa, Artificial Sequence
  Synthetic
DRXYIHP

SEQ ID NO: 15 - protein, 8 aa, Artificial Sequence
  Synthetic
XXXXXXXX

SEQ ID NO: 16 - protein, 5 aa, Artificial Sequence
  Synthetic
DRXYI

SEQ ID NO: 17 - protein, 6 aa, Artificial Sequence
  Synthetic
DRXYIH

SEQ ID NO: 18 - protein, 8 aa, Artificial Sequence
  Synthetic
DRVYVHPF

SEQ ID NO: 19 - protein, 8 aa, Artificial Sequence
  Synthetic
NRVYVHPF

SEQ ID NO: 20 - protein, 11 aa, Artificial Sequence
  Synthetic
APGDRIYVHPF

SEQ ID NO: 21 - protein, 8 aa, Artificial Sequence
  Synthetic
ERVYIHPF

SEQ ID NO: 22 - protein, 8 aa, Artificial Sequence
  Synthetic
DKVYIHPF

SEQ ID NO: 23 - protein, 8 aa, Artificial Sequence
  Synthetic
DRAYIHPF

SEQ ID NO: 24 - protein, 8 aa, Artificial Sequence
  Synthetic
DRVTIHPF

SEQ ID NO: 25 - protein, 8 aa, Artificial Sequence
  Synthetic
DRVYLHPF

SEQ ID NO: 26 - protein, 8 aa, Artificial Sequence
  Synthetic
DRVYIRPF

SEQ ID NO: 27 - protein, 8 aa, Artificial Sequence
  Synthetic
DRVYIHAF

SEQ ID NO: 28 - protein, 8 aa, Artificial Sequence
  Synthetic
DRVYIHPY

SEQ ID NO: 29 - protein, 8 aa, Artificial Sequence
  Synthetic
PRVYIHPF

SEQ ID NO: 30 - protein, 8 aa, Artificial Sequence
  Synthetic
DRVYIHPF

SEQ ID NO: 31 - protein, 8 aa, Artificial Sequence
  Synthetic
DRLYIHPF

SEQ ID NO: 32 - protein, 8 aa, Artificial Sequence
  Synthetic
DRVYLHPF

SEQ ID NO: 33 - protein, 9 aa, Artificial Sequence
  Synthetic
DRVSYIHPF

SEQ ID NO: 34 - protein, 7 aa, Artificial Sequence
  Synthetic
XXXXXXX

SEQ ID NO: 35 - protein, 7 aa, Artificial Sequence
  Synthetic
XXYXHPF

SEQ ID NO: 36 - protein, 7 aa, Artificial Sequence
  Synthetic
RVYGHPF

SEQ ID NO: 37 - protein, 7 aa, Artificial Sequence
  Synthetic
RVYAHPF

SEQ ID NO: 38 - protein, 7 aa, Artificial Sequence
  Synthetic
DRVAIHP

SEQ ID NO: 39 - protein, 7 aa, Artificial Sequence
  Synthetic
DRPYIHP

SEQ ID NO: 40 - protein, 7 aa, Artificial Sequence
  Synthetic
DRKYIHP

SEQ ID NO: 41 - protein, 7 aa, Artificial Sequence
  Synthetic
DRLYIHP

SEQ ID NO: 42 - protein, 8 aa, Artificial Sequence
  Synthetic
DRVYIHPI

SEQ ID NO: 43 - protein, 7 aa, Artificial Sequence
  Synthetic
RVAIHPF

SEQ ID NO: 44 - protein, 7 aa, Artificial Sequence
  Synthetic
RVAIHPF

SEQ ID NO: 45 - protein, 8 aa, Artificial Sequence
  Synthetic
GRVYIHPF

SEQ ID NO: 46 - protein, 7 aa, Artificial Sequence
  Synthetic
RVYLHPF

SEQ ID NO: 47 - protein, 7 aa, Artificial Sequence
  Synthetic
DRYIHPF

SEQ ID NO: 48 - protein, 8 aa, Artificial Sequence
  Synthetic
DXVYIHPF

SEQ ID NO: 49 - protein, 7 aa, Artificial Sequence
  Synthetic
DVYIHPF

SEQ ID NO: 50 - protein, 7 aa, Artificial Sequence
  Synthetic
DRPAIHP

SEQ ID NO: 51 - protein, 7 aa, Artificial Sequence
  Synthetic
DRPAIHP

(19) United States
(12) Patent Application Publication
(10) Pub. No.: US 2014/0205631 A1
(43) Pub. Date: Jul. 24, 2014

(54) Stimulation of vaccination by angiotensin peptides

(71) Applicant: University of Southern California, Los Angeles, CA (US)

(72) Inventors: Robert LARSEN, Los Angeles, CA (US); Kathleen E. RODGERS, Long Beach, CA (US); Gere S. DIZEREGA, San Luis Obispo, CA (US)

(21) Appl. No.: 14/159,973

(22) Filed: Jan. 21, 2014

Related U.S. Application Data
(60) Provisional application No. 61/755,710, filed on Jan. 23, 2013.

(51) Int. Cl. A61K 39/39; A61K 39/21

(52) U.S. Cl. CPC A61K 39/39; A61K 39/21
USPC 424/201.1; 424/204.1; 424/234.1; 424/208.1

(57) ABSTRACT

The present invention provides angiotensin peptide compositions and methods for use of the compositions in vaccination.

### CROSS REFERENCE

This application claims priority to U.S. Provisional Patent Application Ser. No. 61/755,710 filed Jan. 23, 2013, incorporated by reference herein in its entirety.

### BACKGROUND

The acquired immune deficiency syndrome (AIDS) epidemic is sustained largely through sexual transmission of human immunodeficiency virus (HIV). Often, HIV infection appears to be established with just a very few founder viruses. As the virus breaches the mucosal barrier there is a vigorous host immune response. Augmenting the mucosal immunological barrier prior to exposure to HIV by mucosal immunization may prevent transmission through immune exclusion.

### SUMMARY OF THE INVENTION

In a first aspect, the invention provides pharmaceutical compositions, comprising

(a) an amount effective of an immunogen sufficient to elicit an immune response in a mammal;

(b) an amount effective of an angiotensin peptide or salt thereof to stimulate a local immune response in a mammal at a site of application of the composition; and

(c) a pharmaceutically acceptable carrier.

In one embodiment, the immunogen comprises an immunogen selected from the group consisting of a killed viral immunogen, a killed bacterial immunogen, one or more viral proteins or antigenic fragments thereof, and one or more bacterial proteins or antigenic fragments thereof, or combinations thereof. In another embodiment, the immunogen comprises an immunogen selected from the group consisting of a killed human immunodeficiency virus (HIV) immunogen, a killed feline immunodeficiency virus (FIV) immunogen, one or more HIV proteins or antigenic fragments thereof, and one or more FIV proteins or antigenic fragments thereof, or combinations thereof. In a still further embodiment, the vaccine comprises an immunogen selected from the group consisting of HIV-gp120 or antigenic fragments thereof and FIV-p24 or antigenic fragments thereof, or combinations thereof.

In another embodiment, the composition is formulated for mucosal administration.

In a second aspect, the invention provides methods of vaccination, comprising administering to a subject in need of vaccination:

(a) a vaccine; and

(b) an amount effective of an angiotensin peptide or salt thereof to stimulate a local immune response in a mammal at a site of application of the composition;

wherein the vaccine and the angiotensin peptide are administered topically to a mucosa of the subject. In one embodiment, the vaccine and the angiotensin peptide are administered as a single pharmaceutical composition. In another embodiment, vaccine comprises an immunogen selected from the group consisting of a killed viral immunogen, a killed bacterial immunogen, one or more viral proteins or antigenic fragments thereof, and one or more bacterial proteins or antigenic fragments thereof, or combinations thereof. In a further embodiment, the vaccine comprises an immunogen selected from the group consisting of a killed human immunodeficiency virus (HIV) immunogen, a killed feline immunodeficiency virus (FIV) immunogen, one or more HIV proteins or antigenic fragments thereof, and one or more FIV proteins or antigenic fragments thereof, or combinations thereof. In a still further embodiment, the vaccine comprises an immunogen selected from the group consisting of HIV-gp120 or antigenic fragments thereof and FIV-p24 or antigenic fragments thereof, or combinations thereof.

### DETAILED DESCRIPTION OF THE INVENTION

As used herein, the singular forms “a,” “an” and “the” include plural referents unless the context clearly dictates otherwise. “And” as used herein is interchangeably used with “or” unless expressly stated otherwise.

All embodiments within and between different aspects of the invention can be combined unless the context clearly dictates otherwise.

As used herein, the term “about” means+/−5% of the relevant measurement or unit.

In a first aspect, the present invention provides pharmaceutical composition, comprising

(a) an amount effective of an immunogen sufficient to elicit an immune response in a mammal;

(b) an amount effective of an angiotensin peptide or salt thereof, to stimulate a local immune response in a mammal at a site of application of the composition; and

(c) a pharmaceutically acceptable carrier.

The inventors have demonstrated that angiotensin peptides are broadly applicable as vaccine adjuvants, and thus the recited pharmaceutical compositions have broad use, for example, in vaccinations. The pharmaceutical compositions of the invention may include any suitable angiotensin peptide, such as those described in detail below. In one embodiment, the angiotensin peptide comprises or consists of A(1-7), with an amino acid sequence of Asp-Arg-Val-Tyr-Ile-His-Pro (SEQ ID NO: 4).

In one embodiment, the peptides for use in the invention comprise or consist of a sequence of at least four contiguous amino acids of groups R¹-R⁸ in the sequence of general formula I

(SEQ ID NO: 1)
R¹-R²-R³-R⁴-R⁵-R⁶-R⁷-R⁸

wherein R¹ is selected from the group consisting of H, Asp, Glu, Asn, Acpc (1-aminocyclopentane carboxylic acid), Ala, Me²Gly, Pro, Bet, Glu(NH₂), Gly, Asp(NH₂) and Suc, or is absent,

R² is selected from the group consisting of Arg, Lys, Ala, Cit, Orn, Ser(Ac), Sar, D-Arg and D-Lys,

R³ is selected from the group consisting of Val, Ala, Leu, norLeu, Ile, Gly, Lys, Pro, Aib, Acpc and Tyr;

R⁴ is selected from the group consisting of Tyr, Tyr(PO₃)₂, Thr, Ser, homoSer, azaTyr, and Ala;

R⁵ is selected from the group consisting of Ile, Ala, Leu, norLeu, Val and Gly;

R⁶ is selected from the group consisting of His, Arg or 6-NH₂-Phe;

R⁷ is selected from the group consisting of Pro or Ala; and

R⁸ is selected from the group consisting of Phe, Phe(Br), Ile and Tyr, excluding sequences including R⁴ as a terminal Tyr group.

Exemplary AT2 agonists useful in the practice of the invention include the All analogues set forth above subject to the restriction that R⁶ is p-NH₂-Phe.

In a further preferred embodiment of each of the above embodiments (SEQ ID NO: 15),

R¹ is selected from the group consisting of Asp and Glu, or is absent;

R² is selected from the group consisting of Arg, Lys, and Ala;

R³ is selected from the group consisting of Val, Ala, Leu, norLeu, Ile, Gly, Lys, and Pro;

R⁴ is selected from the group consisting of Tyr and homoSer;

R⁵ is selected from the group consisting of Ile, Ala, Leu, norLeu, Val and Gly;

R⁶ is selected from the group consisting of His and Arg;

R⁷ is selected from the group consisting of Pro or Ala; and

R⁸ is selected from the group consisting of Phe, Ile, or is absent.

In alternate embodiments, the peptides comprise or consist of at least five, six, seven, or eight contiguous amino acids of groups R¹-R⁸ in the sequence of general formula I. In a further alternative, the polypeptides consist essentially of a sequence of at least four, five, six, seven, or eight contiguous amino acids of groups R¹-R⁸ in the sequence of general formula I.

Particularly preferred combinations for R¹ and R² are Asp-Arg, Asp-Lys, Glu-Arg and Glu-Lys. Particularly preferred embodiments of this class include the following: AIII or AII(2-8), Arg-Val-Tyr-Ile-His-Pro-Phe [SEQ ID NO:2]; AII(3-8), also known as des1-AIII or AIV, Val-Tyr-Ile-His-Pro-Phe [SEQ ID NO:3]; AII(1-7), Asp-Arg-Val-Tyr-Ile-His-Pro [SEQ ID NO:4]; AII(2-7). Arg-Val-Tyr-Ile-His-Pro [SEQ ID NO:5]; AII(3-7), Val-Tyr-Ile-His-Pro [SEQ ID NO:6]; AII(5-8), Ile-His-Pro-Phe [SEQ ID NO:7]; AII(1-6), Asp-Arg-Val-Tyr-Ile-His [SEQ ID NO:8]; AII(1-5), Asp-Arg-Val-Tyr-Ile [SEQ ID NO:9]; AII(1-4), Asp-Arg-Val-Tyr [SEQ ID NO:10]; and AII(1-3), Asp-Arg-Val. Other preferred embodiments include: Arg-norLeu-Tyr-Ile-His-Pro-Phe [SEQ ID NO:11] and Arg-Val-Tyr-norLeu-His-Pro-Phe [SEQ ID NO:12]. Still another preferred embodiment encompassed within the scope of the invention is a peptide having the sequence Asp-Arg-Pro-Tyr-Ile-His-Pro-Phe [SEQ ID NO:13].

Other preferred embodiments comprise or consist of

SEQ ID NO: 18
Asp-Arg-Val-Tyr-Val-His-Pro-Phe
SEQ ID NO: 19
Asn-Arg-Val-Tyr-Val-His-Pro-Phe
SEQ ID NO: 20
Ala-Pro-Gly-Asp-Arg-Ile-Tyr-Val-His-Pro-Phe
SEQ ID NO: 21
Glu-Arg-Val-Tyr-Ile-His-Pro-Phe
SEQ ID NO: 22
Asp-Lys-Val-Tyr-Ile-His-Pro-Phe
SEQ ID NO: 23
Asp-Arg-Ala-Tyr-Ile-His-Pro-Phe
SEQ ID NO: 24
Asp-Arg-Val-Thr-Ile-His-Pro-Phe
SEQ ID NO: 25
Asp-Arg-Val-Tyr-Leu-His-Pro-Phe
SEQ ID NO: 26
Asp-Arg-Val-Tyr-Ile-Arg-Pro-Phe
SEQ ID NO: 27
Asp-Arg-Val-Tyr-Ile-His-Ala-Phe
SEQ ID NO: 28
Asp-Arg-Val-Tyr-Ile-His-Pro-Tyr
SEQ ID NO: 29
Pro-Arg-Val-Tyr-Ile-His-Pro-Phe
SEQ ID NO: 13
Asp-Arg-Pro-Tyr-Ile-His-Pro-Phe
SEQ ID NO: 30
Asp-Arg-Val-Tyr(PO₃)₂-Ile-His-Pro-Phe
SEQ ID NO: 31
Asp-Arg-norLeu-Tyr-Ile-His-Pro-Phe
SEQ ID NO: 32
Asp-Arg-Val-Tyr-norLeu-His-Pro-Phe
SEQ ID NO: 33
Asp-Arg-Val-homoSer-Tyr-Ile-His-Pro-Phe

Another class of peptides of particular interest in accordance with the present invention are those of the general formula II:

(SEQ ID NO: 34)
R²-R³-R⁴-R⁵-R⁶-R⁷-R⁸

in which R² is selected from the group consisting of H, Arg, Lys, Ala, Orn, Citron, Ser(Ac), Sar, D-Arg and D-Lys;

R³-R⁸ are as defined above, and

excluding sequences including R⁴ as a terminal Tyr group.

A particularly preferred subclass of the compounds of general formula II has the formula:

[SEQ ID NO: 35]
R²-R³-Tyr-R⁵-His-Pro-Phe

wherein R², R³ and R⁵ are as previously defined. Particularly preferred is angiotensin III of the formula Arg-Val-Tyr-Ile-His-Pro-Phe [SEQ ID NO:2]. Other preferred compounds include peptides having the structures Arg-Val-Tyr-Gly-His-Pro-Phe [SEQ ID NO:36] and Arg-Val-Tyr-Ala-His-Pro-Phe [SEQ ID NO:37].

In the above formulas, the standard three-letter abbreviations for amino acid residues are employed. Other residues are abbreviated as follows:

TABLE 1
Abbreviation for Amino Acids

| | |
|---|---|
| Me²Gly | N,N-dimethylglycyl |
| Bet | 1-carboxy-N,N,N-trimethylmethanaminium hydroxide |
| | inner salt (betaine) |
| Suc | Succinyl |
| Phe(Br) | p-bromo-L-phenylalanyl |
| azaTyr | aza-α′-homo-L-tyrosyl |
| Acpc | 1-aminocyclopentane carboxylic acid |
| Aib | 2-aminoisobutyric acid |
| Sar | N-methylglycyl (sarcosine) |
| Cit | Citron |
| Orn | Ornithine |
| NorLeu (Nle) | NorLeucine |
| HomoSer | HomoSerine (isotheronine) |

It has been suggested that All and its analogues adopt either a gamma or a beta turn (Regoli, et al., Pharmacological Reviews 26:69 (1974)). In general, it is believed that neutral side chains in position R³, R⁵ and R⁷ may be involved in maintaining the appropriate distance between active groups in positions R⁴, R⁶ and R⁸ primarily responsible for binding to receptors and/or intrinsic activity. Hydrophobic side chains in positions R³, R⁵ and R⁸ may also play an important role in the whole conformation of the peptide and/or contribute to the formation of a hypothetical hydrophobic pocket.

Appropriate side chains on the amino acid in position R² may contribute to affinity of the compounds for target receptors and/or play an important role in the conformation of the peptide. For this reason, Arg and Lys are particularly preferred as R². Alternatively, R₂ may be H, Ala, Orn, Citron, Ser(Ac), Sar, D-Arg, or D-Lys.

For purposes of the present invention, it is believed that R³ may be involved in the formation of linear or nonlinear hydrogen bonds with R⁵ (in the gamma turn model) or R⁶ (in the beta turn model). R³ would also participate in the first turn in a beta antiparallel structure (which has also been proposed as a possible structure). In contrast to other positions in general formula I, it appears that beta and gamma branching are equally effective in this position. Moreover, a single hydrogen bond may be sufficient to maintain a relatively stable conformation. Accordingly, R³ may suitably be selected from Lys, Val, Ala, Leu, norLeu, Ile, Gly, Pro, Aib, Acpc and Tyr.

With respect to R⁴, conformational analyses have suggested that the side chain in this position (as well as in R³ and R⁵) contribute to a hydrophobic cluster believed to be essential for occupation and stimulation of receptors. Thus, R⁴ is preferably selected from Tyr, Thr, Tyr (PO₃)₂, homoSer, Ser and azaTyr. In this position, Tyr is particularly preferred as it may form a hydrogen bond with the receptor site capable of accepting a hydrogen from the phenolic hydroxyl (Regoli, et al. (1974), supra). It has also been found that R⁴ can be Ala, and that it can be used for cyclization of angiotension peptides.

In position R⁵, an amino acid with a β aliphatic or alicyclic chain is particularly desirable. Therefore, while Gly is suitable in position R⁵, it is preferred that the amino acid in this position be selected from Ile, Ala, Leu, norLeu, and Val.

In another embodiment, in peptides of particular interest in accordance with the present invention R⁶ is His, Arg or 6-NH₂-Phe. The unique properties of the imidazole ring of histidine (e.g., ionization at physiological pH, ability to act as proton donor or acceptor, aromatic character) are believed to contribute to its particular utility as R⁶. For example, conformational models suggest that His may participate in hydrogen bond formation (in the beta model) or in the second turn of the antiparallel structure by influencing the orientation of R⁷. Similarly, it is presently considered that R⁷ should be Pro or Ala in order to provide the most desirable orientation of R⁸. In position R⁸, both a hydrophobic ring and an anionic carboxyl terminal appear to be particularly useful in binding of the analogues of interest to receptors; therefore, Tyr, Ile, Phe(Br), and especially Phe are preferred for purposes of the present invention.

Analogues of particular interest include the following:

TABLE 2
Angiotensin II Analogues

| AII | | |
|---|---|---|
| Analogue | | Sequence |
| Name | Amino Acid Sequence | Identifier |
| Analogue 1 | Asp-Arg-Val-Tyr-Val-His-Pro-Phe | SEQ ID NO: 18 |
| Analogue 2 | Asn-Arg-Val-Tyr-Val-His-Pro-Phe | SEQ ID NO: 19 |
| Analogue 3 | Ala-Pro-Gly-Asp-Arg-Ile-Tyr-Val-His-Pro-Phe | SEQ ID NO: 20 |
| Analogue 4 | Glu-Arg-Val-Tyr-Ile-His-Pro-Phe | SEQ ID NO: 21 |
| Analogue 5 | Asp-Lys-Val-Tyr-Ile-His-Pro-Phe | SEQ ID NO: 22 |
| Analogue 6 | Asp-Arg-Ala-Tyr-Ile-His-Pro-Phe | SEQ ID NO: 23 |
| Analogue 7 | Asp-Arg-Val-Thr-Ile-His-Pro-Phe | SEQ ID NO: 24 |
| Analogue 8 | Asp-Arg-Val-Tyr-Leu-His-Pro-Phe | SEQ ID NO: 25 |
| Analogue 9 | Asp-Arg-Val-Tyr-Ile-Arg-Pro-Phe | SEQ ID NO: 26 |
| Analogue 10 | Asp-Arg-Val-Tyr-Ile-His-Ala-Phe | SEQ ID NO: 27 |
| Analogue 11 | Asp-Arg-Val-Tyr-Ile-His-Pro-Tyr | SEQ ID NO: 28 |
| Analogue 12 | Pro-Arg-Val-Tyr-Ile-His-Pro-Phe | SEQ ID NO: 29 |
| Analogue 13 | Asp-Arg-Pro-Tyr-Ile-His-Pro-Phe | SEQ ID NO: 13 |
| Analogue 14 | Asp-Arg-Val-Tyr(PO₃)₂-Ile-His-Pro-Phe | SEQ ID NO: 30 |
| Analogue 15 | Asp-Arg-norLeu-Tyr-Ile-His-Pro-Phe | SEQ ID NO: 31 |
| Analogue 16 | Asp-Arg-Val-Tyr-norLeu-His-Pro-Phe | SEQ ID NO: 32 |
| Analogue 17 | Asp-Arg-Val-homoSer-Tyr-Ile-His-Pro-Phe | SEQ ID NO: 33 |

Other particularly preferred embodiments include:

1GD Ala4-AII(1-7)
SEQ ID NO: 38
DRVAIHP
2GD Pro3-AII(1-7)
SEQ ID NO: 39
DRPYIHP
5GD Lys3-AII(1-7)
SEQ ID NO: 40
DRKYIHP
9GD NorLeu-AII(1-7)
SEQ ID NO: 41
DR(nor)YIHP
GSD 28 I1e⁸-AII
SEQ ID NO: 42
DRVYIHPI
Ala3aminoPhe6 AII:
SEQ ID NO: 43
RVAIHPF
Ala3-AIII
SEQ ID NO: 44
RVAIHPF
Gly¹-AII
SEQ ID NO: 45
GRVYIHPF
NorLeu⁴-AIII
SEQ ID NO: 46
--RVYnLHPF
Acpc³-AII
SEQ ID NO: 47
DR(Acpc)YIHPF
GSD 37B Orn²-AII
SEQ ID NO: 48
D(Orn)VYIHPF
GSD38B Citron²-AII
SEQ ID NO: 49
D(Citron)VYIHPF
3GD Pro³Ala⁴-AII(1-7)
SEQ ID NO: 50
DRPAIHP
8GD Hydroxy-Pro³-AII(1-7)
SEQ ID NO: 51
DRP(OH)AIHP

In another embodiment, the peptides may be any of those disclosed in US20100055146, incorporated by reference herein in its entirety. In various embodiments, the polypeptide is:

a 4,7-cyclized analog of Angiotensin II (Ang(1-8), or any of its analogues disclosed herein;

a 4,7-cyclized analog of Angiotensin III (Ang(2-8)), or any of its analogues disclosed herein;

a 4,7-cyclized analog of Angiotensin IV (Ang(3-8)), or any of its analogues disclosed herein; or

a 4,7-cyclized analog of Ang(1-7), or any of its analogues disclosed herein.

In another embodiment, the methods comprise administering an agonist of the MAS receptor. Any suitable polypeptide or non-polypeptide agonist of the MAS receptor may be used, including but not limited to A(1-7) and analogues thereof, A779 (D-Ala A(1-7); available from Sigma Chemical Co.) and AVE0991, (see, for example, Pinheiro et al., Hypertension. 2004 October; 44(4):490-6. Epub 2004 Aug. 23).

The polypeptides for use in the present invention may be linear or cyclized in any suitable manner, such as those described in WO2008/018792, including but not limited to polypeptides comprising a thioether bridge between positions 4 and 7, or other positions.

The polypeptides may be recombinantly expressed or chemically synthesized using any suitable techniques, which are well within the level of those of skill in the art.

Any suitable immunogen or combination of immunogens can be used in the compositions of the invention. The immunogen(s) must be capable of stimulating an immune response in any suitable mammal, including but not limited to humans, cattle, pigs, goats, dogs, cats, horses and any other mammalian pet or food animal such as chickens, turkeys, Cornish game hens, pheasants, or ducks.

For example, the immunogen can be a whole or partial product of a virus, bacteria, fungal organism, mycobacteria, parasite (single cell or multiple cell), or derived from a plant, fermentation product (generally produced by yeasts and/or molds) or engineered through chemical synthesis (for example a DNA vaccine antigen). Additionally, the immunogen could be derived from human cells, including but not limited to cancer cells or parts thereof, which could enhance host responses to destroying cancer cells.

In one non-limiting embodiment, the immunogen is a bacteria or antigenic portion thereof. In one embodiment, the bacteria may include, but is not limited to, at least one of: Neisseria meningitides; N. gonorrheoeae; Legionella pneumonia; Vibrio cholerae; Vibrio parahemolyticus, Vibrio volnificans, Streptococcal species including Group A streptococci; Staphylococcus aureus; Staphylococcus epidermidis; Pseudomonas aeruginosa; Corynobacteria diphtheriae; Clostridium spp. including C. perfringens; Eschericia coli; Bacillus anthracis; Bartonella henselae; Bartonella quintana; Coxiella burnetii; Chlamydia pneumonia, C. trachomatis; Mycobacterium leprae; M. tuberculosis, Non-tuberculous mycobacteria; Salmonella species including S. typhi and non-typhoidal strains; Shigella sonnie and other Shigella species; Helicobacter pylori; H. influenza, H. parainfluenzae, Yersinia enterocolitica; Y. pseudotuberculosis; Listeria monocytogenes; Mycoplasma pneumonia; M. urilyticum; Treponema pallidum; Leptospira species; Francisella tularensis; Brucella melitensis; B. aborus, B. suis, Campylobacter jejuni; Enterobacter aerogenes and other enterobacteracea; Proteus mirabilis and other Proteus species; and Klebsiella pneumonia and other Klebsiella species, or combinations thereof.

In another non-limiting embodiment, the immunogen is a vaccine already in general use but would be enhanced by addition of an angiotensin peptide. In one embodiment vaccine the vaccine in use could be the polyvalent influenza vaccine either the killed virus or live virus; the polyvalent pneumococcal vaccine; the polyvalent meningococcal vaccine; the polio vaccine; the multiple vaccine combination measles, mumps and rubella vaccine; or the tetanus and diphtheria vaccine, or combinations thereof.

In another non-limiting embodiment, the immunogen is a fungi or antigenic portion thereof. In one embodiment, the fungi may include, but is not limited to Candida species, such as C. albicans; Aspergilus species; Histoplasma capsulatum; Bastomycosis dermatiditis; Coccidioides immitis or the capsular antigen of Cryptococcus neoformans, or combinations thereof.

In a further non-limiting embodiment, the immunogen is a virus or antigenic portion thereof. In one embodiment, the virus may include, but is not limited to adenovirus; coxsackievirus; Enterocytopathic human orphan viruses (ECHO); hepatitis A, B, C, D or X viruses alone or in combination; the human herpes virus family currently composed of Epstein-Barr virus; herpes simplex; type 1; herpes simplex; type 2; human cytomegalovirus; human herpesvirus; type 8; varicella-zoster virus; human immunodeficiency virus (HIV); influenza viruses; measles virus; mumps virus; parainfluenza virus; respiratory syncytial virus; papillomaviruses; rabies virus; and encephalitis viruses including but not limited to St. Louis encephalitis virus, West Nile virus, Japanese encephalitis virus, and Equine encephalitis viruses, or combinations thereof.

In another non-limiting embodiment, the immunogen is a parasite or antigenic portion thereof. In one embodiment, the parasite may include, but is not limited to trypanosome; haemoprotozoa and parasites capable of causing malaria; amoeba, enteric and systemic cestode; taeniid cestod; enteric coccidian; enteric flagellate protozoa; filarial nematode; gastrointestinal and systemic nematode and hookworm.

In one embodiment, the immunogen is one derived from a retrovirus, including but not limited to a whole retrovirus or portion thereof. Exemplary and non-limiting such retroviruses are human immunodeficiency virus (HIV), feline immunodeficiency virus (FIV), feline leukemia virus (FeLV), bovine leukemia virus, human T-lymphotropic virus (HTLV), and as yet undiscovered retroviruses. Preferably, the immunogens are selected from the group consisting of a killed human immunodeficiency virus (HIV) immunogen, a killed feline immunodeficiency virus (FIV) immunogen, one or more HIV proteins or antigenic fragments thereof, and one or more FIV proteins or antigenic fragments thereof, or combinations thereof.

When the immunogen comprises a viral immunogen, any suitable immunogen from the virus can be used. In one non-limiting embodiment where the immunogen is an HIV immunogen, the immunogen may, for example, comprise gp41 antigen, gp120 antigen, p24 antigen, or gp160 antigen, or combinations thereof.

The pharmaceutical compositions can be formulated for delivery by any suitable route, including but not limited to mucosal, dermal topical applications, or injection by intradermal, transdermal, subcutaneous, intramuscular, intraperitoneal, intraarticular, epidural, intrathecal injection, ingested or taken through a feeding tube, passed or applied by an endoscope to the upper airway, throat, esophagus, upper or lower intestinal track, anus or by vaginal colposcopy, intravenous or intra-arterial infusion or bolus injection, or absorption through epithelial or mucocutaneous linings. In one non-limiting embodiment, the composition is formulated for mucosal administration.

The carrier or diluent may include time delay material, such as glyceryl monostearate or glyceryl distearate alone or with a wax, or other materials well known in the art

In some embodiments, the pharmaceutical compositions are formulated as a gel. In these embodiments, the polypeptide, or salt thereof, may be present in the composition at a concentration of about 0.001% to about 3% on a weight (mg)/volume (ml) basis, or on a weight/weight (mg) basis. In various further embodiments, the polypeptide, or salt thereof, is administered in a pharmaceutical formulation at a concentration of about 0.005% to about 3%; about 0.01% to about 3%; about 0.05% to about 3%; about 0.01% to about 3%; about 0.5% to about 3%; about 1% to about 3%; about 2% to about 3%; about 0.005% to about 2%; about 0.01% to about 2%; about 0.05% to about 2%; about 0.01% to about 2%; about 0.5% to about 2%; about 1% to about 2%; about 0.005% to about 1%; about 0.01% to about 1%; about 0.05% to about 1%; about 0.01% to about 1%; about 0.5% to about 1%; about 0.005% to about 0.75%; about 0.01% to about 0.75%; about 0.005% to about 0.75%; about 0.01% to about 0.75%; about 0.03% to about 1%; about 0.03% to about 0.75%; about 0.03% to about 0.5%; about 0.03% to about 0.25%; about 0.03% to about 0.1%; about 0.03% to about 0.075%; about 0.03% to about 0.05%; and about 0.03%; all on a weight (mg)/volume (ml) basis, or on a weight/weight (mg) basis.

In another embodiment of all aspects of the invention, the pharmaceutically acceptable carrier comprises about 0.5% to about 4% hydroxyethyl cellulose (HEC) on a weight (mg)/volume (ml) basis, or on a weight/weight (mg) basis. In various further embodiments, the pharmaceutically acceptable carrier may comprise about 1% to about 3% HEC, or about 2% HEC, on a weight (mg)/volume (ml) basis, or on a weight/weight (mg) basis.

Any suitable amount effective of the angiotensin peptide (such as A(1-7)) may be used in the compositions, as appropriate for a given use. In various non-limiting embodiments, the angiotensin peptide is present at between about 0.01 mg/ml to about 30 mg/ml; about 0.01 mg/ml to about 10 mg/ml; about 0.1 mg/ml to about 30 mg/ml; about 0.1 mg/ml to about 10 mg/ml; about 1 mg/ml to about 30 mg/ml; and about 1 mg/ml to about 10 mg/ml.

In all aspects of the invention, the polypeptides, or salt thereof may be administered (or present in the pharmaceutical compositions) together with one or more (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The peptides may be administered with a lyoprotectant, e.g. sucrose, sorbitol or trehalose. In certain embodiments, the peptides may be administered with a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the peptides may be administered with a bulking agent, like glycine. In yet other embodiments, the peptides may be administered with a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The peptides may be administered with a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the peptides may be administered with a stabilizer, e.g., a molecule which, when combined with the peptide substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride, paraben, and combinations of methyl paraben and propyl paraben.

In all aspects and embodiments of the invention, suitable acids which are capable of forming salts with the polypeptides include, but are not limited to, inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid and the like. Suitable bases capable of forming salts with the peptides include, but are not limited to, inorganic bases such as sodium hydroxide, ammonium hydroxide, potassium hydroxide and the like; and organic bases such as mono-, di- and tri-alkyl and aryl amines (e.g., triethylamine, diisopropyl amine, methyl amine, dimethyl amine and the like) and optionally substituted ethanol-amines (e.g., ethanolamine, diethanolamine and the like).

The polypeptides or salts thereof can further be derivatized to provide enhanced half-life, for example, by linking to polyethylene glycol. The peptides or salts thereof may comprise L-amino acids, D-amino acids (which are resistant to L-amino acid-specific proteases in vivo), a combination of D- and L-amino acids, and various “designer” amino acids (e.g., β-methyl amino acids, Cα-methyl amino acids, and Nα-methyl amino acids, etc.) to convey special properties.

The polypeptides may be the sole active agent in the pharmaceutical composition, or the composition may further comprise one or more other active agents suitable for the desired treatment. The methods may be used in conjunction with other therapies suitable for treating the relevant disorder.

All embodiments of the compositions, and combinations thereof, can also be used in the methods of the invention.

In a second aspect, the invention provides methods of vaccination, comprising administering to a subject in need of vaccination:

(a) a vaccine; and

(b) an amount effective of an angiotensin peptide or salt thereof to stimulate a local immune response at a site of its administration to the subject;

wherein the vaccine and the angiotensin peptide or salt thereof are administered topically to a mucosa of the subject.

The subject can be any suitable mammalian subject or food animal, as described herein. The subject is one that is at risk of, but not yet suffering from, a disorder caused by the pathogen that the vaccine is directed against. As used herein, “method of vaccination” means to limit development of a disorder caused by the pathogen by causing an immune response prior to exposure to active pathogen, such that future exposure to the pathogen is less likely to result in an active disorder caused by the pathogen.

Any suitable angiotensin peptide or salt thereof may be used in the invention, as disclosed above. In one embodiment, the angiotensin peptide is A(1-7) or a salt thereof. In one embodiment, the vaccine and the angiotensin peptide or salt thereof are administered as a single pharmaceutical composition; in this embodiment, the method may comprise administering a pharmaceutical composition according to any embodiment or combination of embodiments of the invention.

The vaccine may comprise any suitable immunogen as described for the pharmaceutical compositions of the invention. The immunogen(s) must be capable of stimulating an immune response in any suitable mammal, including but not limited to humans, cattle, pigs, goats, dogs, cats, horses and any other mammalian pet or food animal such as chickens, turkeys, Cornish game hens, pheasants, or ducks.

For example, the immunogen can be a whole or partial product of a virus, bacteria, fungal organism, mycobacteria, parasite (single cell or multiple cell), or derived from a plant, fermentation product (generally produced by yeasts and/or molds) or engineered through chemical synthesis (for example a DNA vaccine antigen). Additionally, the immunogen could be derived from human cells, including but not limited to cancer cells or parts thereof, which could enhance host responses to destroying cancer cells.

In one non-limiting embodiment, the immunogen is a bacteria or antigenic portion thereof. In one embodiment, the bacteria may include, but is not limited to, at least one of: Neisseria meningitides; N. gonorrheoeae; Legionella pneumonia; Vibrio cholerae; Vibrio parahemolyticus, Vibrio volnificans, Streptococcal species including Group A streptococci; Staphylococcus aureus; Staphylococcus epidermidis; Pseudomonas aeruginosa; Corynobacteria diphtheriae; Clostridium spp. including C. perfringens; Eschericia coli; Bacillus anthracis; Bartonella henselae; Bartonella quintana; Coxiella burnetii; Chlamydia pneumonia, C. trachomatis; Mycobacterium leprae; M. tuberculosis, Non-tuberculous mycobacteria; Salmonella species including S. typhi and non-typhoidal strains; Shigella sonnie and other Shigella species; Helicobacter pylori; H. influenza, H. parainfluenzae, Yersinia enterocolitica; Y. pseudotuberculosis; Listeria monocytogenes; Mycoplasma pneumonia; M. urilyticum; Treponema pallidum; Leptospira species; Francisella tularensis; Brucella melitensis; B. aborus, B. suis, Campylobacter jejuni; Enterobacter aerogenes and other enterobacteracea; Proteus mirabilis and other Proteus species; and Klebsiella pneumonia and other Klebsiella species, or combinations thereof.

In another non-limiting embodiment, the immunogen is a vaccine already in general use but would be enhanced by addition of an angiotensin peptide. In one embodiment vaccine the vaccine in use could be the polyvalent influenza vaccine either the killed virus or live virus; the polyvalent pneumococcal vaccine; the polyvalent meningococcal vaccine; the polio vaccine; the multiple vaccine combination measles, mumps and rubella vaccine; or the tetanus and diphtheria vaccine, or combinations thereof.

In another non-limiting embodiment, the immunogen is a fungi or antigenic portion thereof. In one embodiment, the fungi may include, but is not limited to Candida species, such as C. albicans; Aspergilus species; Histoplasma capsulatum; Bastomycosis dermatiditis; Coccidioides immitis or the capsular antigen of Cryptococcus neoformans, or combinations thereof.

In a further non-limiting embodiment, the immunogen is a virus or antigenic portion thereof. In one embodiment, the virus may include, but is not limited to adenovirus; coxsackievirus; Enterocytopathic human orphan viruses (ECHO); hepatitis A, B, C, D or X viruses alone or in combination; the human herpes virus family currently composed of Epstein-Barr virus; herpes simplex; type 1; herpes simplex; type 2; human cytomegalovirus; human herpesvirus; type 8; varicella-zoster virus; human immunodeficiency virus (HIV); influenza viruses; measles virus; mumps virus; parainfluenza virus; respiratory syncytial virus; papillomaviruses; rabies virus; and encephalitis viruses including but not limited to St. Louis encephalitis virus, West Nile virus, Japanese encephalitis virus, and Equine encephalitis viruses, or combinations thereof.

In another non-limiting embodiment, the immunogen is a parasite or antigenic portion thereof. In one embodiment, the parasite may include, but is not limited to trypanosome; haemoprotozoa and parasites capable of causing malaria; amoeba, enteric and systemic cestode; taeniid cestod; enteric coccidian; enteric flagellate protozoa; filarial nematode; gastrointestinal and systemic nematode and hookworm.

In one embodiment, the immunogen is one derived from a retrovirus, including but not limited to a whole retrovirus or portion thereof. Exemplary and non-limiting such retroviruses are human immunodeficiency virus (HIV), feline immunodeficiency virus (FIV), feline leukemia virus (FeLV), bovine leukemia virus, human T-lymphotropic virus (HTLV), and as yet undiscovered retroviruses. Preferably, the immunogens are selected from the group consisting of a killed human immunodeficiency virus (HIV) immunogen, a killed feline immunodeficiency virus (FIV) immunogen, one or more HIV proteins or antigenic fragments thereof, and one or more FIV proteins or antigenic fragments thereof, or combinations thereof.

When the immunogen comprises a viral immunogen, any suitable immunogen from the virus can be used. In one non-limiting embodiment where the immunogen is an HIV immunogen, the immunogen may, for example, comprise gp41 antigen, gp120 antigen, p24 antigen, or gp160 antigen, or combinations thereof.

In all embodiments of the methods of the invention, the peptide or salt thereof may be administered by any suitable route, including but not limited to mucosal, dermal topical applications, or injection by intradermal, transdermal, subcutaneous, intramuscular, intraperitoneal, intraarticular, epidural, intrathecal injection, ingested or taken through a feeding tube, passed or applied by an endoscope to the upper airway, throat, esophagus, upper or lower intestinal track, anus or by vaginal colposcopy, intravenous or intra-arterial infusion or bolus injection, or absorption through epithelial or mucocutaneous linings. In one non-limiting embodiment, the peptide is administered mucosally.

In one exemplary embodiment, a gastrointestinal route can be used in which, for example, a gel is applied at the time of upper endoscopy (for example, H. pylori vaccination topically in the upper GI tract) or lower endoscopy (for example, tumor or any of the toxin diseases listed above or even colon cancer).

In one non-limiting embodiment, the inventors have discovered that injection of A(1-7) with, for example, FIV antigens increased mucosal anti-FIV antibodies in the rectum. The injection of the A(1-7) had distal effects not necessarily to the site of administration. Thus, the compositions and formulations can be used, for example, for limiting development of sexually transmitted diseases where the vaccine is given as an injection in a muscle group like the arm or buttock.

In some embodiments, the angiotensin polypeptide, or salt thereof, may be administered in a pharmaceutical formulation at a concentration of about 0.001% to about 3% on a weight (mg)/volume (ml) basis, or on a weight/weight (mg) basis. In various further embodiments, the polypeptide, or salt thereof, is administered in a pharmaceutical formulation at a concentration of about 0.005% to about 3%; about 0.01% to about 3%; about 0.05% to about 3%; about 0.01% to about 3%; about 0.5% to about 3%; about 1% to about 3%; about 2% to about 3%; about 0.005% to about 2%; about 0.01% to about 2%; about 0.05% to about 2%; about 0.01% to about 2%; about 0.5% to about 2%; about 1% to about 2%; about 0.005% to about 1%; about 0.01% to about 1%; about 0.05% to about 1%; about 0.01% to about 1%; about 0.5% to about 1%; about 0.005% to about 0.75%; about 0.01% to about 0.75%; about 0.005% to about 0.75%; about 0.01% to about 0.75%; about 0.03% to about 1%; about 0.03% to about 0.75%; about 0.03% to about 0.5%; about 0.03% to about 0.25%; about 0.03% to about 0.1%; about 0.03% to about 0.075%; about 0.03% to about 0.05%; and about 0.03%; all on a weight (mg)/volume (ml) basis, or on a weight/weight (mg) basis.

In another embodiment of the methods of the invention, the pharmaceutically acceptable carrier comprises about 0.5% to about 4% hydroxyethyl cellulose (HEC) on a weight (mg)/volume (ml) basis, or on a weight/weight (mg) basis. In various further embodiments, the pharmaceutically acceptable carrier may comprise about 1% to about 3% HEC, or about 2% HEC, on a weight (mg)/volume (ml) basis, or on a weight/weight (mg) basis.

Any suitable amount effective of the angiotensin peptide (such as A(1-7)) may be used in the methods of the invention, as deemed appropriate for a given use by an attending physician. In various non-limiting embodiments, the angiotensin peptide is present at between about 0.01 mg/ml to about 30 mg/ml; about 0.01 mg/ml to about 10 mg/ml; about 0.1 mg/ml to about 30 mg/ml; about 0.1 mg/ml to about 10 mg/ml; about 1 mg/ml to about 30 mg/ml; and about 1 mg/ml to about 10 mg/ml.

The methods may include any other embodiments as disclosed in the example that follows. Such embodiments may be used in any combination in the methods of the invention, unless the context clearly dictates otherwise.

### Examples

The studies described below characterize the effect of angiotensin (1-7) when co-administered with the killed feline immunodeficiency virus (Fel-O-Vax FIV) vaccine on the generation of mucosal immunity. We demonstrated a dramatic increase in local secretory IgA immune responses.

### Introduction

The AIDS epidemic is sustained largely through sexual transmission of HIV. Often, HIV infection appears to be established with just a very few founder viruses. As the virus breaches the mucosal barrier there is a vigorous host immune response. Augmenting the mucosal immunological barrier prior to exposure to HIV by mucosal immunization may prevent transmission through immune exclusion. Local generation of secretory IgA antibodies (SIgA) constitutes the largest humoral immune system in the body. SIgA has been demonstrated to effectively block epithelial penetration of HIV and in individuals negative for HIV who live with a HIV-positive partner often appear to be protected by specific SIgA in their genital tract. SIgA in saliva has been shown to cleave gp120 effectively neutralizing the ability of HIV to bind and enter host cells. Certain regions in the gp120 surface protein are constant and required to maintain HIV infectious capability. Antibodies to the gp-120 region have broad neutralizing ability. SIgA antibodies to gp120 would disrupt the CD4 cell binding site complex which is essential for virus-host cell recognition steps and thus would prevent HIV entry into host cells and the resultant viral replication.

The feline immunodeficiency virus (FIV) is a retrovirus which occurs worldwide in domestic cats and can lead to immunodeficiency similar to HIV. FIV shares multiple pathogenic properties with HIV. Both HIV and FIV cause cytopathic effects in lymphocytes leading to CD4+ T-lymphocyte cell depletion and opportunistic infections and both viruses infect T-lymphocytes, employ the CXCR⁴ co-receptor for cell entry (although this is not the primary binding receptor for HIV), and can be transmitted by blood, sexual secretions and through vertical transmission. Thus, the pathobiology of FIV in cats is not dissimilar to that of HIV in humans. HIV infection has been established in a cat model system, making FIV a suitable candidate for evaluating local immune effects on the potential to interrupt “sexual” transmission of this retroviral agent. At present a killed FIV virus vaccine (Fel-O-Vax® FIV) has proven efficacy in preventing disease, but not infection.

We describe our experience employing angiotensin (1-7) in combination with a killed FIV vaccine used as the source of antigen in raising mucosal antibodies to FIV following topical application.

### Methods

Animals: Eleven (11) female outbred cats>24 weeks old that were free of FIV, feline leukemia virus (FeLV) and feline infectious peritonitis virus were employed. The animals were restrained for the application of test articles to mucous membranes and collection of blood and mucosal secretions. Sedation, if necessary, was induced by subcutaneous administration of ketamine hydrochloride (22 mg/kg) and acepromazine maleate (0.1 mg/kg). Euthanasia was accomplished with a lethal overdose of barbiturates. All activities were approved by the Institutional Animal Care and Use Committee and supervised by veterinary staff.

Vaccine Preparation: Fel-O-Vax FIV (Fort Dodge Animal Health, Fort Dodge, Iowa) and A(1-7) was combined in a constant volume of 2% hydroxyethyl cellulose (2% HEC). The range of concentrations of A(1-7) ranged from 0 to 10 mg/mL with a constant 0.33 mL of Fel-O-Vax FIV suspended in each mL of the gel applied for vaccination purposes. Two control animals received intramuscular Fel-O-Vax FIV at baseline, week 3 and week 5. One control animal had mucosal vaccination with Fel-O-Vax alone suspended in the 2% HEC without A(1-7) and one control animal received intramuscular Fel-O-Vax FIV with 0.3 mg/kg of A(1-7) at baseline, week 3 and week 5.

Delivery of angiotensin(1-7) and FIV vaccine to mucosal surfaces: 2% HEC, a viscous gel, was used as the vehicle for the topical delivery of angiotensin(1-7) and FIV vaccine. The topical vaccine was applied to the mucosal surface weekly for 6 consecutive weeks. 1.0 mL was applied to the muzzle for oral mucosal vaccination and 1.0 mL and 0.2 mL was instilled in the rectal and vaginal vaults, respectively for mucosal vaccination at these sites.

Measurement of Local Immune Response: The Weck-Cel Surgical Spear (Windsor Biomedical, Newton, N.H.) was used to obtain secretions. Once inserted, the sponge is allowed to remain in place for 5 minutes. The secretions were extracted by centrifugation with 300 μL of a buffer solution. The buffer solution is prepared by mixing 50 μL of 100× protease inhibitor cocktail and 250 μL of 10% Igepal (Sigma, St Louis, Mo., U.S.A.) with 4.7 ml of phosphate-buffered saline (PBS) containing 0.25% bovine serum albumin (BSA, Fraction V; Sigma). The 100× protease inhibitor cocktail contains 100 μg/mL aprotinin, 500 μg/mL leupeptin, 100 μg/mL bestatin (all Sigma), and 50 μg/mL 4-(2-Aminoethyl)-benzenesulfonyl fluoride hydrochloride (Roche Applied Sciences, U.S.A.) in phosphate buffered saline.

The local immune response in mucosal secretions was evaluated against the homologous FIV-p24 antigen (Cell Biolabs, Inc, San Diego, Calif.) or the heterologous HIV-gp120 antigen (ImmunoDiagnostics, Inc, Woburn, Mass.). Plates were prepared with 50 ng/mL FIV-p24 antigen or with 500 ng/mL HIV-gp120 antigen (Bachem Americans, Inc, Torrance, Calif.) incubated over night at 4° C., washed and the target sample added and incubated overnight again at 4° C. The plates were washed and the indicator goat anti-feline IgG (Accurate Chemical & Scientific Corp., Westbury, N.Y., working titer 1:10,000) or goat anti-feline IgA (Accurate Chemical & Scientific Corp., Westbury, N.Y., working titer 1:1,000) added and incubated for 24 hours at 4° C. Following a final wash, substrate solution was added. The stop solution was added after 15 minutes for the IgG reaction and after 30 minutes for the IgA reaction. Plates were read at 450 nm. A positive titer had a relative absorbance>0.100 above the control sample.

### Results

Intramuscular injection of Fel-O-Vax FIV resulted in high serum IgG titers against FIV-p24 antigen at levels of 1:10,000 to 1:20,000 (Table 1). These responses were durable throughout the eight week study period. Topical vaccination using Fel-O-Vax FIV alone or in combination with A(1-7) produced only low serum IgG antibody titers to FIVp24 antigen in some animals (⅜, 38%) with no discernible dose response effect from increasing dose levels of A(1-7).

In oral secretions, intramuscular injections of Fel-O-Vax FIV induced only low levels of SIgA-FIV-p24 antibodies. SIgA-HIV-gp120 antibodies were found in high concentrations which increased when A(1-7) was combined with the intramuscular injections to even higher titers (Table 2). Comparable levels of SIgA-anti-HIV-gp120 antibody titers could be achieved with topical application of the FIV antigen and A(1-7). The dose response observed with increasing A(1-7) concentrations in the topical vaccine was complex with an initial raise and then decline in SIgA-anti-HIV-gp120 antibody titers as A(1-7) concentrations increased. IgG anti-HIV-gp120 antibodies in oral secretions were found in much lower concentrations than the SIgA-anti-HIV-gp120 antibodies.

SIgA antibodies were not induced in vaginal secretions by intramuscular injection of Fel-O-Vax FIV in the absence of A(1-7) [Table 3]. Topical application of FIV antigen with A(1-7) again induced substantial SIgA-anti-HIV-gp120 antibodies. At lower dose levels of A(1-7) IgG anti-HIV-pg120 antibodies were also elicited, but as higher dose levels of A(1-7) were employed IgG antibody induction declined. Again, only low levels of SIgA against FIV-p24 were observed.

High levels of SIgA anti-HIV-gp120 were found with either parenteral vaccination or topical application of Fel-O-Vax FIV with A(1-7) [Table 4]. Low levels of SIgA-anti-FIV-p24 were found in both methods of vaccination.

### Discussion

Vaccines that act at mucosal surfaces offer several potential advantages: 1) virus could be trapped and inactivated at the mucosal surface prior to spread into the host's cells, 2) locally produced immune responses (external to the host) are expected to be more effective in preventing HIV infection since it would effectively block access to the systemic immune system and avoid the consequences of HIV replication in immune cells within the host, 3) mucosal application of a vaccine is painless, easily accomplished and can be widely distributed to locations with limited medical resources and 4) with A(1-7) used as an adjuvant to stimulate local immune responses it has been possible to stimulate mucosal SIgA while having minimal effect on IgG synthesis. This latter effect may be critical since it is believed that IgG may facilitate entry of HIV into host cells and IgA (the monomeric form) may inhibit IgG antibody-dependent cellular cytotoxicity.

Our studies have demonstrated that induction of SIgA antibody production at the mucosal surface following A(1-7)+FIV immunization is sustained for the two month period of observation. It is noteworthy that each mucosal surface (vaginal, oral and rectal) responded differently to the topical application of our vaccine materials suggesting that differing dose levels of A(1-7) and antigen may be used at each mucosal site. It is interesting that parenteral Fel-O-Vax FIV and A(1-7) appears to increase trafficking of cells to the vaginal mucosal surface.

Progress toward an effective HIV vaccine has been stymied for a quarter century. Combining A(1-7), as an adjuvant to enhance mucosal immune function is a novel approach.

TABLE 1
Serum antibody titers

| Vaccine | Serum IgG anti-FIVp24 antigen Titers¹ | | | |
|---|---|---|---|---|
| Strategy | Week 2 | Week 4 | Week 6 | Week 8 |
| IM | N.T.² | 1:10,000 | N.T. | 1:10,000 |
| IM | N.T. | N.T. | 1:10,000 | 1:10,000 |
| IM + A(1-7) | N.T. | 1:20,000 | 1:40,000 | 1:20,000 |
| Topical only | N.T. | 1:20 | 1:20 | 1:20 |
| 0.1 A(1-7) | N.T. | Negative | N.T. | Negative |
| 0.2 A(1-7) | N.T. | Negative | N.T. | 1:20 |
| 0.3 A(1-7) | N.T. | 1:20 | N.T. | 1:20 |
| 0.4 A(1-7) | N.T. | Negative | N.T. | Negative |
| 5.0 A(1-7) | Negative | Negative | Negative | Negative |
| 7.5 A(1-7) | Negative | Negative | Negative | Negative |
| 10.0 A(1-7) | Negative | Negative | Negative | Negative |

1:20 was the lowest dilution tested.
N.T. is not tested.

TABLE 2
Oral Antibody Titers

| | | | | | SIgA anti- | IgG anti- |
|---|---|---|---|---|---|---|
| Vaccine | SIgA Anti-FIV-p24¹ | | | | HIV-gp120² | FIV-p24² |
| Strategy | Week 2 | Week 4 | Week 6 | Week 8 | Week 8 | Week 8 |
| IM | 1:64 | 1:8 | 1:4 | Undilute | 1:512 | 1:4 |
| IM | Negative | Undilute | 1:4 | Negative | 1:256 | Negative |
| IM + A(1-7) | Negative | Undilute | 1:8 | Undilute | 1:4,096 | 1:4 |
| Topical only | Negative | Undilute | Negative | Negative | 1:16³ | Negative |
| 0.1 A(1-7) | Negative | Negative | Undilute | Negative | 1:128 | Negative |
| 0.2 A(1-7) | Negative | Negative | Negative | Negative | 1:128 | Negative |
| 0.3 A(1-7) | Negative | Negative | Negative | Negative | 1:2,048 | Negative |
| 0.4 A(1-7) | 1:64 | Undilute | Negative | 1:4 | 1:128 | 1:16 |
| 5.0 A(1-7) | Undilute | 1:8 | 1:8 | 1:4 | 1:16³ | 1:16 |
| 7.5 A(1-7) | 1:4 | Negative | 1:8 | 1:128 | Negative | 1:128 |
| 10.0 A(1-7) | Negative | Negative | Negative | Negative | Negative | Negative |

Undilute was the lowest dilution tested.
1:4 was the lowest dilution tested. Elution factor 4-fold
Sample tested was from week 6.

TABLE 3
Vaginal Secretory Antibody Titers

| | | | | | SIgA Anti- | IgG Anti- |
|---|---|---|---|---|---|---|
| Vaccine | SIgA Anti-FIV-p24¹ | | | | HIV gp120² | HIV gp120² |
| Strategy | Week 2 | Week 4 | Week 6 | Week 8 | Week 8 | Week 8 |
| IM | 1:8 | 1:4 | Undilute | Undilute | Negative | 1:9,600 |
| IM | Negative | Negative | Negative | Negative | Negative³ | Not tested |
| IM + A(1-7) | Negative | Undilute | Undilute | 1:4 | 1:9,600 | Not tested |
| Topical only | 1:4 | Negative | Undilute | Negative | Negative | Negative |
| 0.1 A(1-7) | Undilute | Undilute | 1:16 | 1:4 | Negative | Negative |
| 0.2 A(1-7) | Undilute | 1:4 | 1:8 | Undilute | 1:4,800 | 1:76,800 |
| 0.3 A(1-7) | 1:64 | 1:8 | Undilute | Negative | 1:2,400 | 1:4,800 |
| 0.4 A(1-7) | 1:16 | 1:64 | 1:64 | 1:16 | 1:9,600 | 1:2,400 |
| 5.0 A(1-7) | Negative | Negative | Negative | Negative | Negative | Negative |
| 7.5 A(1-7) | Negative | Negative | Negative | Negative | 1:1,200 | Negative |
| 10.0 A(1-7) | Negative | 1:16 | Negative | Negative | 1:38,400 | Negative |

Undilute was the lowest dilution tested.
1:4 was the lowest dilution tested. Elution factor 300-fold
Sample tested was from week 6.

TABLE 4
Rectal Antibody Titers

| | | | | | SIgA |
|---|---|---|---|---|---|
| | | | | | Anti-HIV |
| Vaccine | SIgA Anti-FIV-p24¹ | | | | gp120² |
| Strategy | Week 2 | Week 4 | Week 6 | Week 8 | Week 8 |
| IM | Undilute | Negative | Negative | Negative | 1:19,200 |
| IM | Not | 1:8 | Negative | Undilute | 1:1,200³ |
| | Tested | | | | |
| IM + A(1-7) | Not | Undilute | Negative | Undilute | 1:1,200³ |
| | Tested | | | | |
| Topical | Undilute | Negative | Negative | Negative | Negative³ |
| only | | | | | |
| 0.1 A(1-7) | Undilute | 1:64 | Undilute | Undilute | 1:9,600 |
| 0.2 A(1-7) | Undilute | 1:16 | Negative | Negative | 1:1:2,400 |
| 0.3 A(1-7) | Undilute | Undilute | Negative | Negative | 1:19,200 |
| 0.4 A(1-7) | 1:4 | Undilute | Undilute | 1:4 | 1:9,600 |
| 5.0 A(1-7) | 1:4 | Negative | Negative | Negative | 1:1,200³ |
| 7.5 A(1-7) | Negative | Negative | 1:8 | Negative | 1:38,400³ |
| 10.0 A(1-7) | Negative | Negative | Negative | Negative | 1:2,400³ |

Undilute was the lowest dilution tested.
1:4 was the lowest dilution tested. Elution factor 300-fold
Sample tested was from week 6.

We claim:

1. A pharmaceutical composition, comprising
(a) an amount effective of an immunogen sufficient to elicit an immune response in a mammal;
(b) an amount effective of an angiotensin peptide or salt thereof to stimulate a local immune response in a mammal at a site of application of the composition; and
(c) a pharmaceutically acceptable carrier.

2. The pharmaceutical composition of claim 1, wherein the immunogen comprises an immunogen selected from the group consisting of a killed viral immunogen, a killed bacterial immunogen, one or more viral proteins or antigenic fragments thereof, and one or more bacterial proteins or antigenic fragments thereof, or combinations thereof.

3. The pharmaceutical composition of claim 1, wherein the immunogen comprises an immunogen selected from the group consisting of a killed human immunodeficiency virus (HIV) immunogen, a killed feline immunodeficiency virus (FIV) immunogen, one or more HIV proteins or antigenic fragments thereof, and one or more FIV proteins or antigenic fragments thereof, or combinations thereof.

4. The pharmaceutical composition of claim 1, wherein the immunogen comprises a an immunogen selected from the group consisting of HIV-gp120 or antigenic fragments thereof and FIV-p24 or antigenic fragments thereof, or combinations thereof.

5. The pharmaceutical composition of claim 1, wherein the composition is formulated for mucosal administration.

6. The pharmaceutical composition of claim 1, wherein the pharmaceutically acceptable carrier comprises between about 0.5% and about 4% hydroxyethyl cellulose (HEC).

7. The pharmaceutical composition of claim 1, wherein the pharmaceutically acceptable carrier comprises about 2% HEC.

8. The pharmaceutical composition of claim 1, wherein the angiotensin peptide is present at between about 0.01 mg/ml and about 30 mg/ml.

9. The pharmaceutical composition of claim 1, wherein the angiotensin peptide is angiotensin 1-7 (A(1-7)) or a salt thereof.

10. The pharmaceutical composition of claim 1, wherein the angiotensin peptide comprises at least four contiguous amino acids of
(a) groups R¹-R⁸ in the sequence of general formula I
(SEQ ID NO: 1)
R¹-R²-R³-R⁴-R⁵-R⁶-R⁷-R⁸
wherein R¹ is selected from the group consisting of H, Asp, Glu, Asn, Acpc (1-aminocyclopentane carboxylic acid), Ala, Me²Gly, Pro, Bet, Glu(NH₂), Gly, Asp(NH₂) and Suc, or is absent,
R² is selected from the group consisting of Arg, Lys, Ala, Cit, Orn, Ser(Ac), Sar, D-Arg and D-Lys,
R³ is selected from the group consisting of Val, Ala, Leu, norLeu, Ile, Gly, Lys, Pro, Aib, Acpc and Tyr;
R⁴ is selected from the group consisting of Tyr, Tyr(PO₃)₂, Thr, Ser, homoSer, azaTyr, and Ala;
R⁵ is selected from the group consisting of Ile, Ala, Leu, norLeu, Val and Gly;
R⁶ is selected from the group consisting of His, Arg or 6-NH₂-Phe;
R⁷ is selected from the group consisting of Pro or Ala; and
R⁸ is selected from the group consisting of Phe, Phe(Br), Ile and Tyr, excluding sequences including R⁴ as a terminal Tyr group; or
(b) groups R²-R⁸ in the sequence of general formula II R²-R³-R⁴-R⁵-R⁶-R⁷-R⁸ (SEQ ID NO: 34)
in which R² is selected from the group consisting of H, Arg, Lys, Ala, Orn, Citron, Ser(Ac), Sar, D-Arg and D-Lys;
R³-R⁸ are as defined above, and
excluding sequences including R⁴ as a terminal Tyr group.

11. A method of vaccination, comprising administering to a subject in need of vaccination:
(a) a vaccine; and
(b) an amount effective of an angiotensin peptide or salt thereof to stimulate a local immune response in a mammal at a site of application of the composition;
wherein the vaccine and the angiotensin peptide are administered topically to a mucosa of the subject.

12. The method of vaccination of claim 11, wherein the vaccine and the angiotensin peptide are administered as a single pharmaceutical composition.

13. The method of claim 11, wherein the vaccine comprises an immunogen selected from the group consisting of a killed viral immunogen, a killed bacterial immunogen, one or more viral proteins or antigenic fragments thereof, and one or more bacterial proteins or antigenic fragments thereof, or combinations thereof.

14. The method of claim 11, wherein the vaccine comprises an immunogen selected from the group consisting of a killed human immunodeficiency virus (HIV) immunogen, a killed feline immunodeficiency virus (FIV) immunogen, one or more HIV proteins or antigenic fragments thereof, and one or more FIV proteins or antigenic fragments thereof, or combinations thereof.

15. The method of claim 11, wherein the vaccine comprises an immunogen selected from the group consisting of HIV-gp120 or antigenic fragments thereof and FIV-p24 or antigenic fragments thereof, or combinations thereof.

16. The method of claim 11, wherein the vaccine and the angiotensin peptide are administered in a formulation comprising between about 0.5% and about 4% hydroxyethyl cellulose (HEC).

17. The method of claim 11, wherein the vaccine and the angiotensin peptide are administered in a formulation comprising about 2% HEC.

18. The method of claim 11, wherein the vaccine and the angiotensin peptide are topically administered at an oral mucosa of the subject.

19. The method of claim 11, wherein the angiotensin peptide is administered at between about 1 mg/ml and about 10 mg/ml.

20. The method of claim 11, wherein the angiotensin peptide is A(1-7) or a salt thereof.

21. The method of claim 11, wherein the angiotensin peptide comprises at least four contiguous amino acids of
(a) groups R¹-R⁸ in the sequence of general formula I
(SEQ ID NO: 1)
R¹-R²-R³-R⁴-R⁵-R⁶-R⁷-R⁸
wherein R¹ is selected from the group consisting of H, Asp, Glu, Asn, Acpc (1-aminocyclopentane carboxylic acid), Ala, Me²Gly, Pro, Bet, Glu(NH₂), Gly, Asp(NH₂) and Suc, or is absent,
R² is selected from the group consisting of Arg, Lys, Ala, Cit, Orn, Ser(Ac), Sar, D-Arg and D-Lys,
R³ is selected from the group consisting of Val, Ala, Leu, norLeu, Ile, Gly, Lys, Pro, Aib, Acpc and Tyr;
R⁴ is selected from the group consisting of Tyr, Tyr(PO₃)₂, Thr, Ser, homoSer, azaTyr, and Ala;
R⁵ is selected from the group consisting of Ile, Ala, Leu, norLeu, Val and Gly;
R⁶ is selected from the group consisting of His, Arg or 6-NH₂-Phe;
R⁷ is selected from the group consisting of Pro or Ala; and
R⁸ is selected from the group consisting of Phe, Phe(Br), Ile and Tyr, excluding sequences including R⁴ as a terminal Tyr group; or
(b) groups R²-R⁸ in the sequence of general formula II R²-R³-R⁴-R⁵-R⁶-R⁷-R⁸ (SEQ ID NO: 34)
in which R² is selected from the group consisting of H, Arg, Lys, Ala, Orn, Citron, Ser(Ac), Sar, D-Arg and D-Lys;
R³-R⁸ are as defined above, and
excluding sequences including R⁴ as a terminal Tyr group.
